(19) United States
(12) Patent Application Publication
(10) Pub. No.: US 2010/0010308 A1
(43) Pub. Date: Jan. 14, 2010

(54) Hygiene protector for endoscopes, endoscope with hygiene protector, and method for fitting a hygiene protector onto an endoscope

(76) Inventors: Michael Braun, Backnang (DE); John S. Geis, Bad Zwischenahn (DE)

Correspondence Address: Mark J Pandiscio;Pandiscio & Pandiscio, 470 Totten Pond Road, Waltham, MA, 02451-1914, US

(21) Appl. No.: 11/990,567

(22) PCT Filed: Aug. 17, 2005
(86) PCT No.: PCT/DE2005/001450

§ 371 Date: Aug. 28, 2009

(51) Int. Cl. A61B 1/00; A61B 1/12

(52) U.S. Cl. 600/121

(57) ABSTRACT

The invention relates to a hygiene protection for endoscopes having at least one working channel (14), wherein the hygiene protection (2) comprises on its distal end a cap (15) with a working channel tube (17) arranged thereon and a protective sheath (21) connected to the cap (15). The hygiene protection is characterized in that the working channel tube (17) is arranged at the cap (15) with a distance to the circumference of the cap (15). Furthermore an endoscope (1) and a method for applying a hygiene protection (2) onto an endoscope (1) are described.

The present invention relates to hygiene protection for an endoscope, to an endoscope with hygiene protection as well as to a method for applying a hygiene protection to an endoscope.

Both examinations for diagnostic purposes as well as minor surgical interventions can be performed in endoscopy by application of endoscopes. For therapeutic applications a working channel is provided in the endoscope in addition to the optics. Through the working channel for example biopsy probes may be sampled. Endoscopes have to be cleaned thoroughly, as they are inserted into body cavities of the human body during treatment. The more versatile the endoscope can be used, the more complex the layout of the endoscope becomes, whereby the cleaning of the endoscope is rendered difficult. In particular with endoscopes having optical elements, such as for example a photoconductor and lights for illuminating the area which is to be examined, it is necessary to provide a scavenging channel, through which the optical elements can be cleaned with air and/or water during the application. Also this channel has to be cleaned after usage of the endoscope, as this channel comes in contact with body fluids. Likewise, also the so called working channel, through which biopsy probes and superfluous secretion as well as residual tissue can be extracted, is in contact with body fluids, so that also this channel has to be subjected to intense cleaning after each usage.

Due to their positioning within the endoscope, in particular in the endoscope shaft, these channels are difficult to access and, hence, have to be subjected to extensive cleaning.

In order to address this problem, the prior art suggests the utilization of protective sheaths for endoscopes. In WO 2004 060 149 A1 for example, a hygiene protection for endoscopes is suggested which consists of a cover closed at the distal end and penetrable by optical information at least at the face and which can be rolled up like a condom in axial direction of the endoscope. In addition, the hygiene protection has one or several functional channels running parallel to the endoscope which terminate in an open fashion at the distal end of the cover. The functional channels are arranged such that these are positioned between the outside of the endoscope and the inside of the cover.

By integrating the functional channels, which may for example serve for biopsy probe sampling and/or can be used for the scavenging agent for the optical elements, into the cover, the contamination of the endoscope shaft and of the channels arranged therein can be prevented. With this hygiene protection the functional channels which are provided at the hygiene protection will be used instead of the channels which are provided in the endoscope. One disadvantage this type of hygiene protection entails is, that the endoscope will have to be bent during application, in order to be able to follow the course of body cavities and to observe and treat these cavities. The functional channels are being compressed or stretched, when the endoscope is bent, as these channels are guided on the outside of the endoscope shaft. In case of stretching, the channels may even tear off from the distal end of the cover and contaminated body fluids may, thus, enter into the cover and contaminate the endoscope. In addition, as the functional channels are guided along the endoscope shaft, they may be damaged or occluded during advance through body cavities of small diameter.

Furthermore, the channels which are introduced between the endoscope and the cover, lead to an increased friction when applying the hygiene protection onto as well as when removing or stripping off the hygiene protection from the endoscope. Finally, wrinkles are formed in the protective cover due to the outer arrangement of the functional channels. The handling and the hygiene of the hygiene protection will be impaired due to this wrinkling.

The invention is based on the problem of providing a possibility which allows for easy and reliable elimination of contamination of an endoscope during endoscopy.

According to a first aspect this problem is solved by a hygiene protection for endoscopes having at least one working channel, wherein the hygiene protection comprises on its distal end a cap with a working channel tube arranged thereon and a protective sheath connected to the cap. The hygiene protection is characterized in that the working channel tube is arranged at the cap with a distance to the circumference of the cap.

According to the present invention the face part of the hygiene protection is referred to as the cap. The face part is preferably made of a hollow cylinder which is closed at its distal end. If not indicated differently, reference to the cap refers to the face thereof. It is also within the scope of the invention that the cap is shaped as a flat disk, which represents the face of the hygiene protection.

According to the present invention, the channel of the endoscope through which instruments for example for sampling of tissue probes (biopsy) or for carrying out smaller surgeries can be introduced, is referred to as the working channel. Furthermore, the working channel also serves for evacuating of body fluids. According to the invention, the working channel tube replaces the working channel for these purposes. The working channel is located within the endoscope, that means inside of the endoscope shaft.

The working channel tube provided on the hygiene protection can be fixed at an opening in the cap which corresponds to the inner diameter of the working channel tube or can extend through an opening in the cap which corresponds to the outer diameter of the working channel tube up to the distal surface of the cap and may possibly extend slightly beyond. The working channel tube can be attached to the cap or can be made integrally with the cap.

As the working channel tube is provided in a distance to the circumference of the cap of the hygiene protection, it is positioned in a small distance to the centre point of the cap. The fiber of the endoscope which is subject to the smallest forces when the endoscope is bent, runs through the centre point and will hereinafter also be referred to as the neutral fiber. The smaller the distance to the neutral fiber, the less the working channel tube will be stretched or compressed when the endoscope is bent. A tearing off of the working channel tube from the cap can thus be avoided.

According to a preferred embodiment the working channel tube is arranged on the cap at the position, which corresponds to the position of the working channel of an endoscope inserted into the protective sheath of the hygiene protection. Thereby, the working channel of the endoscope can serve as a guide for the working channel tube und a separate provisioning of a groove or a different guide for the working channel tube into the endoscope can be omitted. Thereby, the hygiene protection can be used for conventional endoscopes having a working channel without necessitating a modification of the endoscope. Furthermore, the guiding of the working channel tube in the working channel of the endoscope is of advantage, as the working channel tube is protected by the endoscope against forces acting from the outside, in particular against forces directed radially, which may occur when the endoscope is led through a body cavity of small diameter. Therefore, the material of the working channel tube may posses a smaller wall thickness and strength than what would be necessary with a tube guided on the outside. As the hygiene protection has to be disposed after each usage, the material costs and the need for deposition can be minimized by the smaller material thickness of the tube wall of the working channel tube.

Furthermore, the positioning of the working channel tube at the position of the working channel provides a protection against rotation between the hygiene protection and the endoscope. The orientation of the hygiene protection, in particular of the cap, to the distal face of the endoscope is of particular importance, as generally only a part of the cap is designed for the transmission of optical waves, in particular light waves. If such a cap, which is generally not made to be rotationally symmetrical, rotates relative to the endoscope, at least part of the optical elements of the endoscope may be covered and the examination or surgery may be hindered.

Finally, the application of the hygiene protection is facilitated by the positioning of the working channel tube in a position corresponding to the position of the working channel. While with known covers according to the prior art, the operating surgeon had to observe the alignment of the cap with the optical elements of the endoscope when applying the cover, with the hygiene protection according to the invention such an alignment procedure can be omitted.

According to a preferred embodiment, a spraying head is arranged in the cap or integrated therein. The spraying head at least proximally extends in axial direction of the hygiene protection beyond the cap. The spraying head may also extend distally beyond the cap. It is, however, also possible that the spraying head terminates within the cap and does thus not extend in the distal direction beyond the cap.

The direction from the distal end to the proximal end of the hygiene protection is referred to as the proximal direction. The direction from the proximal end to the distal end of the hygiene protection is referred to as the distal direction.

If the spraying head extends in the distal direction beyond the cap, in this area an aperture directed towards the face of the cap and through which scavenging agents, such as water or air, can be fed to the area of the optical elements may be provided. If the spraying head terminates within the cap, it is arranged immediately adjacent to the area of the optical elements, so that also in this embodiment scavenging agent can be applied to this area.

The projection in the proximal direction, in contrast, serves for connecting to a supply of scavenging agent. In particular, with a preferred arrangement of the spraying head, wherein it is spaced from the circumference of the cap, the proximal projection can be aligned with the scavenging channel of the endoscope. In this case, the projection in the proximal direction serves as the entry of the spraying head into the scavenging channel of the endoscope. An additional protection against rotation between the hygiene protection and the endoscope is provided by the entry in the scavenging channel. In addition, the projection seals the transition from the scavenging channel to the spraying head and thus prevents entering of the scavenging agent into the space between the distal end of the endoscope and the cap of the hygiene protection.

According to a preferred embodiment, the proximal end of the spraying head reaches into the distal end of a scavenging channel of an endoscope inserted into the hygiene protection and terminates within the scavenging channel. This means that the length of the proximal projection of the spraying head is essentially smaller than the length of the protective sheath of the hygiene protection. According to the invention it is also possible that the spraying head extends over the entire length of the spraying channel, that means that the proximal projection is designed as a tube. Different than with the working channel, this design is not preferred with the scavenging channel due to its utilization. A bridging between the distal and of the endoscope and the inner side of the cap by means of the spraying head is sufficient for the spraying channel. The applying of the hygiene protection is facilitated, as the spraying head is so short that it terminates within the scavenging channel. It will in particular not be necessary to push a second tube into a channel of the endoscope. In addition, the material costs are lower with a short spraying head, which as described above, is advantageous for the hygiene protection of the invention being a product for one-time usage.

In particular with a spraying head terminating within the scavenging channel, a valve is preferably provided at the proximal end of the spraying head. This valve which is a check valve having an occluding member can for example be a duckbill or dome valve. Even though the supply of scavenging agent to the spaying head can be ensured, an entering of contaminated body liquids or other liquids carrying germs can be prevented with such a valve. The scavenging channel thus does not have to be cleaned after using the endoscope.

The protective sheath of the hygiene protection can have various designs. The protective sheath may for example be a cover made of silicon, latex or a different liquid proof material, wherein this material may be present in a rolled-up state before being applied to the endoscope. In this case, the working channel tube is inserted into the working channel and the cover, which is attached to the cap, is present in a rolled-up state like a condom. For applying the cover onto the endoscope, the cover will be rolled off over the endoscope with the working channel tube being arranged therein, so that the endoscope is completely covered. For removing the endoscope, the cover will be rolled up again.

A further alternative for the design of the protective sheath is, for example, a cover, which is applied via vacuum technology. In this case, the material of the cover may for example be polyethylene, polypropylene, polyurethane, silicon or latex.

The protective sheath is attached to the cap of the hygiene protection and after insertion of the endoscope loosely lies against the endoscope shaft. Subsequently the cover is slightly pulled by hand, so that the entire endoscope is covered. By means of a syringe and/or a pump vacuum is created, which pulls the cover tight and firm against the endoscope surface. In order to remove the cover again, air is introduced by means of a syringe and/or a pump. Thereby, the cover will loosen again from the endoscope and the endoscope can be removed.

A further option of the design of the protective cover is the usage of a so called memory-cover, that means of a cover having shape-memory-effect. Preferably, a cover having a temperature dependent shape-memory-effect will be used. The material may for example be plastic, which reacts on heat and cold. Upon heating, the plastic will expand and will contract upon influence of cold.

In this case, the cover will be manually stretched during insertion of the endoscope, so that the entire endoscope is covered. By influence of cold, for example cold water, the cover will contract. In order to remove the endoscope again, the cover may be stretched by application of heat, for example warm water.

Furthermore a coiled cover can be used as a protective sheath. In this case, the protective cover consists of a tape or a band. The tape or band may consist of plastics such as for example polyurethane, polypropylene or polytetrafluoroethylene (PTFE).

In this case, the protective cover will be coiled up to the proximal end of the endoscope shaft after insertion of the working channel tube into the endoscope so that the entire endoscope shaft will be covered. The coiled cover may be coated at its inner side with an adhesive for improved hold.

Even though these described embodiment of the protective cover, wherein it only consists of one layer, fall within the scope of the present invention, according to a preferred embodiment, the cover comprises at least two layers. The layers each have a tube shape and are arranged within each other. With a design having two layers, it becomes possible to combine material properties of two different materials. In particular, the inner layer may determine the shape of the protective sheath and the outer layer may serve for sealing to prevent contact of the endoscope to contaminated liquids.

According to a preferred embodiment, at least one layer of the protective sheath is designed such that it effects a reduction of diameter of the protective sheath upon axial elongation. Such a layer may in particular be realized as a lattice. As the protective sheath consists of two layers, such a lattice, which by itself would not provide the desired protection, can be used and the additional layer can be impermeable to the entry of air and liquid. The second, outer layer can for example consist of latex or silicon.

It is particularly preferable that the layer, which carries out the change of cross section upon change in length is elastic. This means that the reduction in cross section upon decreasing of the length of the layer is reversed. This is also referred to as elastic self-expanding effect. The utilization of such an elastic layer has the advantage, that the protective sheath on the one hand can easily be applied onto the endoscope and on the other hand can also easily be removed there from. It is particularly advantageous if the diameter of the layer in the relaxed state is larger than in the tensed state and in the relaxed state is larger then the outer diameter of the endoscope. Thereby, the distal end of the endoscope can be inserted into the protective sheath and can be advanced up to the cap of the hygiene protection. No friction forces between the protective sheath, in particular the inner layer and the endoscope, have to be overcome.

Once the part of the endoscope, that means in particular at least the part of the endoscope shaft, which is to be inserted into the body cavity, is enclosed by the protective sheath, an elongation of the layer can be achieved by pulling and thereby the diameter of the protective sheath can be decreased, until it is adjacent the endoscope. Preferably, the protective sheath has a clamp device for fixation of the protective sheath in this position. As the clamp device is provided on the protective sheath, any conventional endoscope can be covered with the hygiene protection. The clamp device, which applies a radial force onto the proximal end of the protective sheath applied onto the endoscope, may for example be a Velcro tape, which may be wrapped around the protective sheath and can thus be fastened. If the clamp device is loosened after usage, the length of the protective sheath will decrease due to its elasticity and the diameter of the protective sheath will regain its original size. In this state, the endoscope can easily be removed from the protective sheath without the endoscope coming into contact with the outside of the protective sheath.

According to one embodiment, the at least two layers are only connected, in particular attached, to each other at the cap and at the proximal end of the protective sheath. Thereby on one side, the production of the hygiene protection will be significantly facilitated and the manufacturing costs will be further lowered. Also material, in particular adhesives can be saved with this embodiment. In particular if a lattice is being used as one of the layers, it may occur, if the further layers are adhered over the entire length, that increasing the distance between the lattice bars will strain the layer which is attached thereto too much and may rip. If for example an elastic foil is being used as additional layer and this layer is only attached at the proximal and distal end to the lattice, the elastic foil may lie on the lattice over the entire length and due to the large number of contact points with the lattice a shifting of the foil relative to the lattice can be prevented. For this purpose, the diameter of the layer in the relaxed state is chosen to be smaller than the diameter of the lattice in the relaxed state.

The cap of the hygiene protection according to the invention preferably comprises a viewing area, which is covered by a foil. The area, which when using the endoscope is positioned over the optical elements, for example light transmitting and luminous elements, is referred to as the viewing area. This area has to be made of a material which transmits optical waves. The viewing area can thus be made of a glass plate, a plastic plate or the like. It has, however, proven that when using materials for the viewing area, which have a certain thickness, reflections occur at the viewing area, which complicate the observation of the area of the body cavity to be viewed or make it impossible to view this area. According to the present invention liquid may be introduced to bridge the space, if a glass plate or a plastic plate is being used.

Particularly preferably a foil is used in the viewing area. According to the invention material having a very small material thickness, for example in the range of 0.01 mm to 0.5 mm, is referred to as foil. It has proven, that when using a foil a reflection of the light does not occur and the introduction of an optical liquid can thus be omitted. The material for the foil may for example be polyethylene, polypropylene, polyurethane or plexiglass.

At the proximal end of the working channel tube according to the invention a valve may be attached, preferably a valve having a side inlet. This valve will be applied after insertion of the working channel tube into the working channel. By means of such a valve it will be possible to insert instruments, such as for the sampling of biopsy probes as well as for sucking off liquids from the area in front of the cap. In these two cases the working channel has to be connected to a corresponding inlet or to a suction device. This is realised by the valve with side inlet which is preferably provided. The side inlet will also be referred to as the side port of the valve. Herein the side inlet of the valve is connected to a suction device. The main channel of the valve, which is sealed at its proximal end, for example, by a membrane, serves for introducing medical instruments. In the duct between the side inlet of the valve and the suction device, which hereinafter will also be referred to as the suction duct, a suction valve is provided, through which the suction from the area in front of the cap of the hygiene protection can be controlled.

Thereby the working channel tube and the suction duct are exclusively formed by components of the hygiene protection. The suction channel which is provided within the endoscope is thus not utilised and cleaning of this suction channel can be omitted.

According to one embodiment a conical extension is provided at the protective sheath at its proximal end. By this conical elongation, which may consist of plastic, for example of polypropylene or polyamide, also the area of the endoscope between the side inlet or side port of the endoscope and the grip of the endoscope can be covered and protected from contamination. The conical extension can be followed by a cover, which can be pulled over the grip of the endoscope and can thereby also prevent contamination of the endoscope grip, of the manipulation wheel and of the suction- and scavenging valve.

Preferably, the valve for connection to the proximal end of the working channel tube is provided at the conical extension of the protective sheath. After application of the conical elongation onto the endoscope, the valve can be connected to and attached with the working channel tube which is guided through the working channel.

According to a further aspect, the invention relates to an endoscope with a hygiene protection, which is characterized in that it comprises a hygiene protection according to the present invention. Preferably the endoscope is designed such that the hygiene protection comprises a cap which is located in the immediate vicinity of the distal end of the endoscope, a working channel tube is guided through the working channel and that the length of the working channel tube is larger than the length of the working channel. The working channel extends from the distal end of the endoscope shaft to the side inlet or side port of the endoscope.

In addition to the advantages of the working channel tube guided in the working channel, which have already been described, the endoscope according to the invention has the advantage that the working channel tube can be guided out of the proximal side of the endoscope due to the large length of the working channel tube. Thereby on one hand a connection between the inlet location for medical instruments and the place of extraction in front of the cap of the hygiene protection can entirely be formed by a part of the hygiene protection, in particular the working channel tube. In addition, the valve provides for the possibility of externally connecting the working channel tube to the suction device.

Preferably a valve having a side inlet is attached to the proximal end of the working channel tube and the side inlet is connected to the suction valve of the endoscope. The side inlet of the valve is referred to as the side port of the valve.

According to one embodiment, air is present between the distal end of the endoscope and the cap of the hygiene protection during utilization of the endoscope. This can, in particular, be realized with the endoscope according to the invention by using a foil as material for the viewing window. In the preferred embodiment the foil lays on the optical elements in immediate contact. The foil may for example be adhered to the face of the cap from the inside over an aperture. Thereby a reflection on the viewing window can be prevented without having to introduce an optical liquid.

According to a last aspect, the present invention relates to a method of applying a hygiene protection on an endoscope. The method is characterized in that the proximal end of a working channel tube of the hygiene protection is inserted into the distal end of the working channel, is advanced through the working channel until the proximal end of the working channel tube extends beyond the proximal end of the working channel. When inserting the endoscope into the hygiene protection the latter is not yet contaminated. Insofar the surgeon can hold the hygiene protection after threading of the working channel tube into the working channel with one hand at the cap and can move the endoscope further.

After insertion of the endoscope shaft into the protective sheath, the latter will be stretched in axial direction until its diameter corresponds to the outer diameter of the endoscope shaft and is affixed in this position at the proximal end of the protective sheath at the endoscope shaft.

The proximal end of the working channel tube is preferably inserted into a valve, through which a connection of a suction device and an inlet for medical instruments is present.

Advantages and features which are described with reference to the hygiene protection also apply—where applicable—to the inventive endoscope and the inventive method, respectively, as well as vice versa.

The invention will hereinafter be described again in more detail with reference to the enclosed figures, wherein:

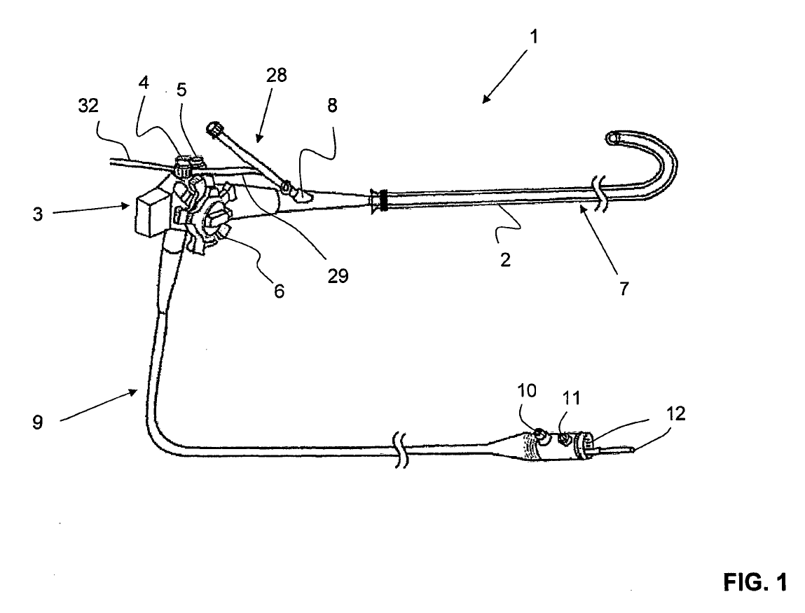

FIG. 1: shows a schematic perspective view of an embodiment of the endoscope according to the invention with a hygiene protection according to the invention;

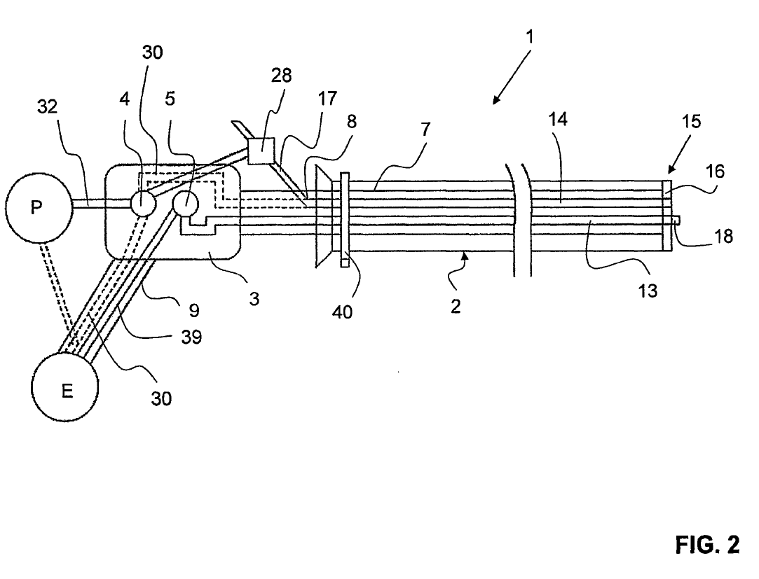

FIG. 2: shows a schematic sectional view of an embodiment of the endoscope according to the invention with a hygiene protection according to the invention;

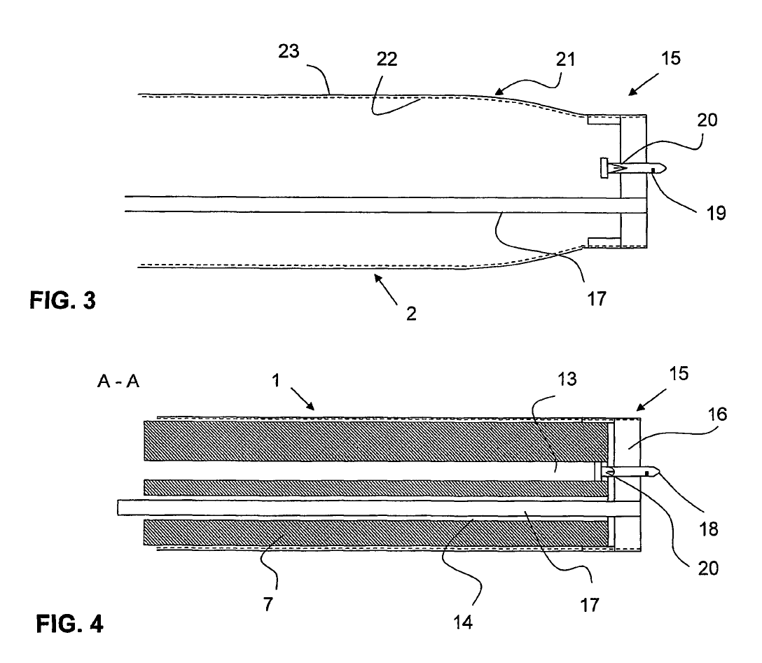

FIG. 3: shows a schematic sectional view through the distal area of an embodiment of a hygiene protection according to the invention;

FIG. 4: shows a schematic sectional view through the distal area of an embodiment of an endoscope according to the invention with a hygiene protection according to the invention;

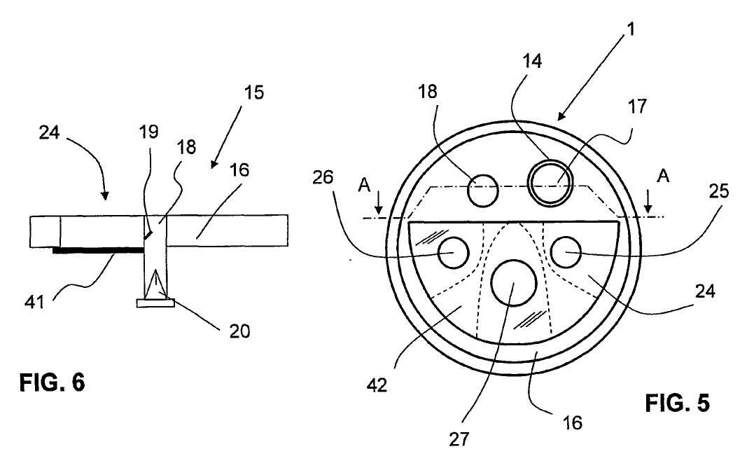

FIG. 5: shows a schematic front view of an embodiment of the endoscope according to the invention with a hygiene protection according to the invention;

FIG. 6: shows a schematic sectional view of a cap of a hygiene protection according to the invention;

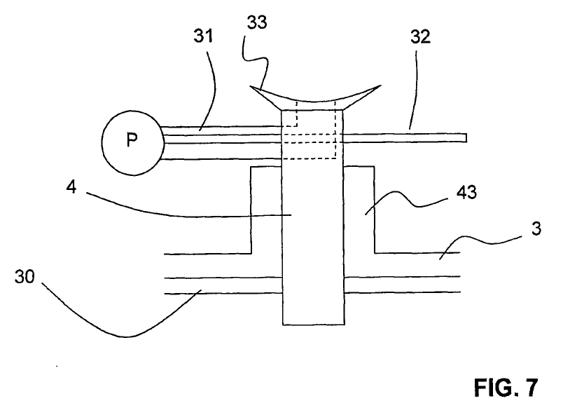

FIG. 7: shows a schematic sectional view of an embodiment of the suction valve according to the invention; and

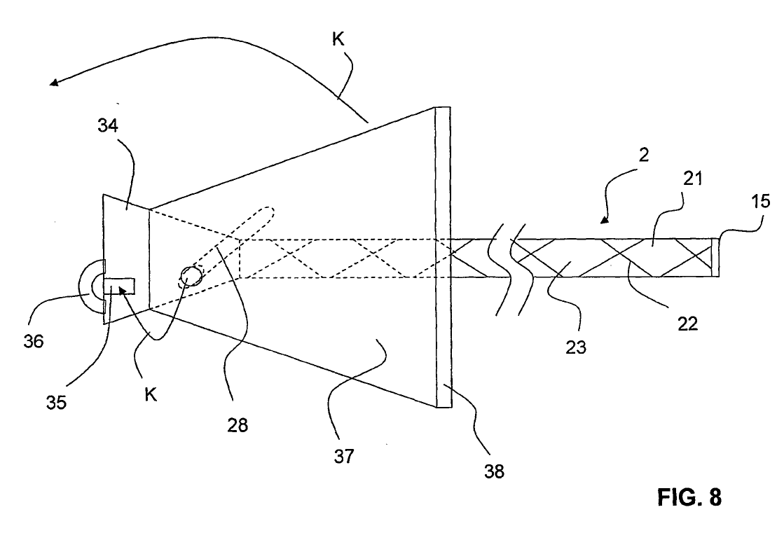

FIG. 8: shows a schematic view of a further embodiment of the hygiene protection according to the invention.

In FIG. 1 an embodiment of the endoscope 1 according to the invention with a hygiene protection 2 according to the invention is shown. The endoscope 1 has a grip 3, where a suction valve 4 and a scavenging valve 5 are provided. In the depicted view only the actuation button of the valves 4 and 5, respectively, which is arranged on top of the grip 3 are visible. On the side the grip 3 has a manipulation wheel 6, by means of which the shaft 7 of the endoscope 1 can be moved. The hygiene protection 2 according to the invention is applied onto the endoscope shaft 7. An inlet 8 which is also referred to as the side port of the endoscope 1 for insertion of medical instruments (not shown) is provided at the distal end of the endoscope shaft 7. At the bottom of the grip 3 a connection duct 9 for connection the endoscope with the unit E for operating the endoscope 1 (see FIG. 2) is provided. A water and air duct, a suction duct as well as optical ducts run through the connection duct 9. The water and air duct exits the connection duct 9 at the end which is connected to unit E laterally and is followed by a water-air-connector 10. At this end of the connection duct 9 also a suction connection 11 is provided at the side which represents the exit of the suction duct in the connection duct 9. At the axial end of the connection duct 9 connectors 12 for the optical elements are provided.

A valve 28 with a side inlet or side port 29 is provided at the side port 8 of the endoscope 1. The functioning of the valve will be described later in detail.

The inner design of the endoscope 1 will hereinafter be described with reference to FIG. 2. A scavenging channel 13 extends from the scavenging valve 5 in the grip 3 of the endoscope 1 over the length of the endoscope shaft 7. A working channel 14 extends parallel to the scavenging channel 13 over the length of the endoscope shaft 7. This working channel 14 on one hand serves for guiding instruments to the distal end of the endoscope shaft 7 and on the other hand serves for sucking off of secretion and tissue remainders. The working channel extends from the side port 8 of the endoscope 1 to the distal end of the endoscope shaft 7. In order to be able to also use this working channel 14 for suction, with common endoscopes a suction channel 30 extends through the proximal end of the endoscope shaft 7, the grip 3 and the connecting duct 9. This suction channel 30 is indicated in FIG. 2 by the dashed line and transitions at the side port 8 into the working channel 14.

The hygiene protection 2 according to the invention is applied onto or into this endoscope 1, respectively, the design of which can in particular be derived from FIG. 3. The hygiene protection 2 consists of a cap 15 which essentially has is cup-shaped. The face of the cap 15 is essentially represented by a disc 16. The disc 16 is penetrated by a working channel tube 17 and is firmly attached thereto. In the depicted embodiment, the distal open end of the working channel tube 17 terminates in the distal end of the disc 16. In addition, a spraying head 18 is provided in the disc 16. In the depicted embodiment, this spraying head 18 extends beyond the distal side and the proximal side of the disc 16. At the distal side a spraying aperture 19 is provided at the side of the spraying head 18 and the distal end of the spraying head 18 is closed. A check valve 20 which is represented by a duckbill valve in FIG. 3 is provided at the proximal end of the spraying head 18.

A protective sheath 21 is attached to the cap 15 at its circumference. The protective sheath 21 consists of two layers 22 and 23, wherein the inner layer 22 is formed by an elastic lattice which upon increase of the length shows a decrease in diameter. In the depicted embodiment the outer layer 23 is a latex foil or a latex tube, respectively. At the cap 15 the protective sheath 21 has the diameter of the cap 15 and expands from there in axial direction to a maximal diameter. This diameter is larger then the outer diameter of an endoscope 1 to be inserted into the hygiene protection 2, in particular larger then the diameter of the endoscope shaft 7. As shown in FIG. 1 and 2 a clamp device, which in the depicted embodiment is a Velcro tape 40, is provided at the proximal end of the protective sheath 21. By means of the Velcro fastener a protective sheath 21 which has been pulled to a desired length at which the diameter of the protective sheath 21 corresponds to the outer diameter of the endoscope shaft 7, that means is adjacent to it, can be fixated to the endoscope 1. It has proven that a Velcro tape 40 wrapped around the protective sheath 21 applies sufficient force to also hold the protective sheath 21 in position when the endoscope 1 is being used.

In FIG. 4 an embodiment of the hygiene protection 2 is shown in a position where it has been applied to the endoscope shaft 7. FIG. 4 shows a sectional view through the endoscope 1 along the sectional line A-A in FIG. 5. The working channel tube 17 is inserted into the working channel 14 of the endoscope 1 and the spraying head 18 extends into the scavenging channel 13 of the endoscope 1. The embodiment of the hygiene protection 2 essentially corresponds to embodiment shown in FIG. 3. In FIG. 4 a dome valve is, however, provided as a check valve 20 for occluding of the scavenging channel 13. The protective sheath 21 is adjacent to the outside of the endoscope shaft 7.

In FIG. 5 a front view of the face of the endoscope 1 is shown with a hygiene protection 2 applied thereto. In this view the open distal end of the working channel tube 17 in the working channel 14 and the closed distal end of the spraying head 18 can be seen. Furthermore a viewing window 24 is provided in the disc 16 of the cap 15. In the depicted embodiment his viewing window essentially has the shape of a half-circle. A foil 41 is arranged within the viewing window 24, and covers the viewing window. The optical elements 25, 26 and 27 of the endoscope 1 can be seen through the viewing window 24. These elements are in particular lights 25 and 26 as well as a photoconductor 27, through which images of the area in front of the disc 16 can be transmitted to the grip 3 and from there to an image processing facility (not shown). The foil 41 is preferably attached to the inside of the disc 16. In the viewing window 24 bars 42 can be provided additionally, which cover the viewing window 24 in areas where none of the optical elements 25, 26 and 27 lie at the distal end of the endoscope 1. The bars 42 are indicated in FIG. 5 with dashed lines and can be part of the disc 16, that means can be integrally formed with the disc 16. The disc 16 obtains an additional stability due to the bars 42 and the areas which are to be covered by the foil 41 are reduced. Alternatively, it is also possible to only place small openings into the disc 16 which correspond to the positions of the optical elements 25, 26 and 27 and have a larger diameter then these elements 25, 26 and 27. The openings can be covered with the foil 41.

In FIG. 6 an embodiment of the disc 16 of the hygiene protection 2 is shown. In this embodiment the spraying head 18 only extends proximally beyond the disc 16. Distally the spraying head 18 terminates flush with the surface of the disc 16 or may even be proximally offset by a certain amount against the surface. With this embodiment the spraying head 18 is arranged immediately abutting to the viewing window 24 and the spraying aperture 19 is directed thereto. Also in this embodiment, the attachment of the foil 41 is realized by attaching, for example adhering, onto the inside of the disc 16.

The guiding of the working channel tube 17 according to the invention will hereinafter be described in more detail with reference to FIGS. 1, 2 and 7.

When applying a hygiene protection 2 onto or into an endoscope 1, respectively, the working channel tube 17 is inserted into the working channel 14 in the endoscope shaft 7 from the distal end. At the side port 8 of the endoscope 1 the working channel tube 17 reaches into this side port 8 and thereby occludes the suction channel 30 which runs in the grip 3 and the connection duct 9. A valve 28 is attached to the proximal end of the working channel tube 17. A side port 29 of this valve 18 in return is connected with the suction valve 4 of the grip 3. FIG. 7 shows a preferred embodiment of such a suction valve 4. The suction valve 4 is embedded in the valve seat 43 which is provided at the grip 3. The lower portion of the suction valve 4 occludes the suction channel 30 in the grip 3. It is, however, also possible that the suction valve 4 has a smaller length and the suction channel 30 thus remains open. This is not crucial because of the working channel tube 17 being inserted into the working channel 14, as the working channel tube 17 already occludes the suction channel 30 at the side port 8.

In the upper part of the suction valve 4, which extends upwards beyond the valve seat 43 for the suction valve 4, a suction duct 32, which is connected at one end to the side port 29 of the valve 28 and at the other end to a pump P, penetrates the suction valve 4. Furthermore an essentially L-shaped channel 31 is provided in the suction valve 4. This channel reaches from the suction valve 4 to the pump P. The channel 31 transitions into a vertical part in the suction valve 4 which extends to the upper side of the suction valve 4. The suction valve 4 is bowl shaped at this upper side and thus forms a finger rest 33. If the surgeon places his finger onto the rest 33, the suction generated by the pump P exclusively acts on the suction duct 32. If, in contrast, the channel 31 is not closed, the suction will act on the suction duct 32 and on the channel 31, wherein the essential suction power will take act via the channel 31 due to its larger diameter and will thus suck in ambient air.

Due to this design, the guiding of the suction duct 32 will entirely be carried out outside of the grip 3 or other parts of the endoscope 1. The suction duct 32, the suction valve 4 and the side port 29 of the valve 28 form part of the hygiene protection 2 and thus are product of one-time usage. With this embodiment of the suction valve 4 it can be inserted into the valve seat 43 which is already provided at the grip 3 and the operation of the endoscope 1 will—as with common endoscopes—be possible with one hand. In addition, there is no need for change of habits for the surgeon.

In FIG. 8 an additional embodiment of the hygiene protection 2 is shown. In the distal area it has the same design as the hygiene protections 2 which have already been described. In the depicted embodiment a conical extension 34 follows the protective sheath 21, which may consist of plastic foil or latex. Due to the conical shape, the proximal end of the endoscope shaft 7 may be enclosed within the extension 34. At the proximal end of the conical extension 34 an aperture 35 is provided, through which the side port 8 of the endoscope 1 can be guided. For fixation of the protective sheath 21, a clamp 36, which may be a fabric ribbon or a metal clamp and which is at least on one side detachably attached to the conical extension 34 is provided. A cover 37 is distally displaced to the aperture 35 provided at the conical extension 34. The cover has a larger diameter than the largest diameter of the conical extension 34. At the proximal end of the cover 37 a clasp 38, which may for example be a tie cord, is provided. Furthermore, the valve 28 is attached to the cover 37.

If the cover 37 is flipped over in the direction which is indicated with K in FIG. 8, it can enclose the grip 3 and a part of the connection duct 9 and can be closed thereon by means of the clasp 38. Thereby, the manipulation wheel 6 at the grip 3 is protected from contamination. This is of importance as the surgeon has to actuate the medical instruments which are guided up to and beyond the distal end of the endoscope 1 and become in contact with body fluids with the same hand that he uses to operate the manipulation wheel 6. Due to the cover 37 also the cleaning of the grip 3 and of the manipulation wheel 6 can be omitted.

The dimensions and the material of the cover 37 are chosen such that it is still possible for the surgeon to operate the manipulation wheel 6.

With the present invention it will thus be possible to reuse an endoscope 1 after its usage again without a cleaning step and yet to reliably prevent contamination of the subsequent patient.

1. Hygiene protection for endoscopes having at least one working channel (14), wherein the hygiene protection (2) comprises on its distal end a cap (15) with a working channel tube (17) arranged thereon and a protective sheath (21) connected to the cap (15), characterized in that the working channel tube (17) is arranged at the cap (15) with a distance to the circumference of the cap (15).

2. Hygiene protection according to claim 1, characterized in that the working channel tube (17) is arranged at the position, which corresponds to the position of the working channel (14) of an endoscope (1) inserted into the protective sheath (21) of the hygiene protection (2).

3. Hygiene protection according to claim 1, characterized in that a spraying head (18) is arranged in the cap (15), which at least proximally extends beyond the cap (15) in axial direction of the hygiene protection (2).

4. Hygiene protection according to claim 3, characterized in that the spraying head (18) has a valve (20) at its proximal end.

5. Hygiene protection according to claim 3, characterized in that the spraying head (18) is arranged at the cap (15) with a distance to the circumference of the cap (15).

6. Hygiene protection according to claim 3, characterized in that the proximal end of the spraying head (18) extends into the distal end of a scavenging channel (13) of an endoscope (1), which is inserted into the protective sheath (21) of the hygiene protection (2), and in that the spraying head (18) terminates within the scavanging channel (13).

7. Hygiene protection according to claim 1, characterized in that the protective sheath (21) comprises at least two layers (22, 23).

8. Hygiene protection according to claim 7, characterized in that at least one layer (22) of the protective sheath causes a reduction of diameter of the protective sheath (21) upon axial elongation.

9. Hygiene protection according to claim 8, characterized in that the layer (22) is a grid.

10. Hygiene protection according to claim 7, characterized in that the at least two layers (22, 23) are only attached to each other at the cap (15) and at the proximal end of the protective sheath (21).

11. Hygiene protection according to claim 1, characterized in that the cap (15) comprises a viewing area (24), which is covered by a foil (41).

12. Hygiene protection according to claim 1, characterized in that a valve (28), preferably a valve with a side port (29), is attached at the proximal end of the working channel tube (17).

13. Hygiene protection according to claim 12, characterized in that the side port (29) is connected to a suction valve (4), which is insertable into a valve seat (43) of an endoscope (1) inserted into the protective sheath (21) of the hygiene protection (2).

14. Hygiene protection according to claim 1, characterized in that a conical extension (34) is attached to the protective sheath (21).

15. Hygiene protection according to claim 14, characterized in that a valve (28) is provided at the conical extension (34) of the protective sheath (21) for connecting to the working channel tube (17).

16. Endoscope with hygiene protection, characterized in that the endoscope comprises a hygiene protection (2) comprising a cap (15), which is located in the immediate vicinity of the distal end of the endoscope, a working channel tube (17) is guided through a working channel (14) of the endoscope and the length of the working channel tube (17) is greater than the length of the working channel (14).

17. (canceled)

18. Endoscope according claim 16, characterized in that a valve (28) with a side port (29) is attached at the proximal end of the working channel tube (17) and in that the side port (29) is connected to the suction valve (4) of the endoscope (1).

19. Endoscope according to claim 16, characterized in that air is present between the distal end of the endoscope (1) and the cap (15) of the hygiene protection (2).

20. Method of applying a hygiene protection (2) on an endoscope (1), characterized in that the proximal end of a working channel tube (17) of the hygiene protection (2) is inserted into the distal end of the working channel (14) of the endoscope (1), is being pushed through the working channel (14), until the proximal end of the working channel tube (17) extends beyond the proximal end of the working channel (14).

21. Method according to claim 20, characterized in that a protective sheath (21) of a hygiene protection (2) is stretched in axial direction after the insertion of an endoscope shaft (7) into the protective sheath (21), until the diameter of the protective sheath (21) corresponds to the outer diameter of the endoscope shaft (7) and in that the protective sheath (21) is affixed in this position to the endoscope shaft (7) at the proximal end of the protective sheath (21).

22. Method according to claims 20, characterized in that the proximal end of the working channel tube (17) is being inserted into a valve (28), by means of which a connection to a suction valve (4) of the endoscope (1) and an inlet for medical devices is provided.
